(19) United States
(12) Patent Application Publication
(10) Pub. No.: US 2011/0293740 A1
(43) Pub. Date: Dec. 1, 2011

(54) TREATMENT SYSTEM AND METHOD FOR OSTEOPENIA AND OSTEOPOROSIS USING NON-SYNTHETIC BIO-AVAILABLE COMPOUNDS

(76) Inventor: Karan Y. Baucom, Overland Park, KS (US)

(21) Appl. No.: 13/116,892

(22) Filed: May 26, 2011

Related U.S. Application Data
(60) Provisional application No. 61/348,309, filed on May 26, 2010.

(51) Int. Cl. A61K 33/42; A61K 31/569; A61K 31/565; A61K 31/566; A61K 31/714; A61K 31/525; A61K 31/51; A61K 31/455; A61K 31/675; A61K 31/519; A61K 33/18; A61K 31/555; A61K 33/22; A61K 33/14; A61K 33/34; A61K 33/04; A61K 33/32; A61P 19/10; A61P 19/08; A61B 19/02; A61K 31/198

(52) U.S. Cl. 424/602; 514/567; 514/171; 514/11.9; 514/52; 514/251; 514/276; 514/356; 514/89; 514/249; 424/667; 514/188; 424/657; 424/677; 424/601; 424/638; 424/709; 424/639; 206/438

(57) ABSTRACT

A treatment system and method for osteopenia and osteoporosis using non-synthetic bio-available hormones, vitamins, minerals, and agents may be customized by varying the dosage and formulation of the components to maximize therapeutic effect, bioavailability, and patient comfort and compliance with their treatment method, the treatment system including a storage unit including a determined number of applications of hormones, minerals, and vitamins of appropriate dosages for treating the patient with osteopenia and osteoporosis.

This application claims priority in U.S. Provisional Patent Application No. 61/348,309, filed on May 26, 2010, which is incorporated herein by reference.

### BACKGROUND OF THE INVENTION

1. Technical Field

The present disclosed technology relates generally to the treatment of bone disorders, and in particular a method for the treatment of osteopenia and osteoporosis using non-synthetic bio-available compounds.

2. Background

Disease and aging of the human body has a profound effect upon the psychology and physical wellbeing of people. The cumulative effect of long-term disease decreases the quality of life. Aging is accompanied by the degradation of body systems and structures. In particular, degenerative conditions of the skeletal system such as a decrease in bone mass and bone health substantially impair the physical abilities of people suffering from disease or age-related conditions.

Degenerative conditions of the bone can make individuals susceptible to bone fractures, bone pain, and other complications. Two significant degenerative conditions of the bone are osteopenia and osteoporosis. Osteopenia is a condition of the bone characterized by a reduction in bone mass due to the loss of bone at a rate greater than new bone growth. Osteopenia manifests in bone having a mineral density lower than normal peak bone mineral density, but not as low as found in osteoporosis. Osteoporosis is a progressive disease characterized by a gradual bone weakening due to demineralization of the bone. Osteoporosis manifests in bones that are thin and brittle making them more susceptible to breaking. Hormone deficiencies related to menopause in women, and hormone deficiencies due to aging in both sexes contribute to degenerative conditions of the bone. In addition, insufficient dietary uptake of minerals essential to bone growth and maintenance are significant causes of bone loss.

Compounds for the treatment of osteopenia or osteoporosis include pharmaceutical preparations that induce bone growth or retard bone demineralization, or mineral complexes that supplement the diet in an effort to replenish lost bone minerals. Low levels of estrogen in women, and low levels of androgen in men are the primary hormonal deficiencies that cause osteoporosis in the respective sexes. Other hormones such as the thyroid hormones, progesterone, and testosterone contribute to bone health. As such, the aforementioned hormonal compounds have been developed synthetically, or extracted from non-mammalian sources, and compounded into therapies for treating osteoporosis. Mineral supplement preparations containing iodine, zinc, manganese, boron, strontium, vitamin D3, calcium, magnesium, vitamin K, phosphorous, and copper have been used to supplement insufficient dietary uptake of such minerals. However, long-term hormonal therapies have undesirable side effects such as increased cancer risk. Moreover, therapies using many synthetic or non-mammalian hormones have additional undesirable side effects, such as an increased risk of cardiovascular disorders, neurological disorders, or the exacerbation of pre-existing conditions.

Effective treatment of osteopenia or osteoporosis requires a careful assessment of the patient's needs, available compounds and modes of administering such compounds that can be adjusted based upon the individual patient's response. Hormones are metabolized partially by cytochrome P450 enzymes. Metabolism of hormones used to treat osteopenia or osteoporosis may be affected by other medications taken by a patient that are inducers or inhibitors of P450 enzymes. As a result, the plasma concentrations of treating hormones may be insufficient to provide a therapeutic effect, or may change the therapeutic profile of the hormone. Patient physiological conditions may dictate use of a particular mode of administration in order to optimize therapeutic effect while administering the lowest effective dose for the shortest duration of treatment. Furthermore, a patient may have a preference for, and respond better to, a particular mode of administration of an available compound notwithstanding the unavailability of a particular compound administered with the preferred modality. Therefore, effective therapies for the treatment of osteopenia or osteoporosis optimize management of the disease by employing characteristics that maximize patient benefit, minimize undesirable side effects, and ensure patient compliance.

For the foregoing reasons, there is a need for a therapy system and method for treating osteopenia and osteoporosis using non-synthetic or bio-available components, quality mineral supplements, and agents in a manner that maximizes treatment benefits, minimizes undesirable side effects, and ensures patient compliance.

Heretofore there has not been available a system and method for the treatment of osteopenia and osteoporosis with the advantages and features of the disclosed subject matter.

### SUMMARY

A treatment system and method for osteopenia and osteoporosis using non-synthetic bio-available hormones, vitamins, minerals, and agents may be customized by varying the dosage and formulation of the components to maximize therapeutic effect, bioavailability, and patient comfort and compliance with their treatment method.

The hormones used for treatment include, but are not limited to thyroid hormones (e.g., thyroxine, or triiodothyronine), progesterone, calcitonin, estrogen (e.g., estradiol), dehydroepiandrosterone (DHEA), and natural testosterone. The vitamins used for treatment include vitamin K (e.g., menaquinone-4 (MK-4), and or menaquinone-7 (MK-7)), vitamin D3 (e.g., cholecalciferol, or calcitrol), and vitamin B Complex (e.g., thiamine (B1), riboflavin (B2), niacin (B3), pyridoxine (B6), folic acid (B9), or cobalamins (B12)). The minerals used for treatment include, but are not limited to iodine or iodide, zinc picolate, manganese, boron, strontium chloride, phosphorous, copper, calcium (e.g., calcium citrate, calcium gluconate, or calcium oxalate), magnesium (e.g., magnesium glycinate, or magnesium sulphate), and calcium hyroxyapatite. The hormones, vitamins, minerals, and agents may be formulated in a variety of dosage forms, including, but not limited to liquids (e.g., oral solutions, nasal sprays, or suspensions), powders, articles for transdermal absorption (e.g., transdermal patch, impregnation matrices, gels, or creams), and ingested articles (e.g., capsules, lozenges, or tablets).

A method embodying the present invention comprises diagnosis of a patient presenting signs of, or suffering from, osteopenia or osteoporosis, and evaluation of a treatment plan for treating a patient using the system described above. A treatment plan is selected by the physician and patient and modified as needed to maximize the therapeutic effect, and bioavailability of the hormones, vitamins, minerals, and agents in order to maximize patient comfort and compliance with the treatment method to effectively manage their osteopenia or osteoporosis.

### BRIEF DESCRIPTION OF THE DRAWINGS

The drawings constitute a part of this specification and include exemplary embodiments of the disclosed subject matter illustrating various objects and features thereof, wherein like references are generally numbered alike in the several views.

FIG. 1 is a plan view of tri-fold wallet-style booklet packaging for dispensing patches, gels, lozenges, and packets containing treatments for osteopenia and osteoporosis embodying the principles of the disclosed subject matter.

FIG. 2 is a plan view of a bi-fold booklet packaging with a drawstring pouch for dispensing sprays, patches, gels, lozenges, and packets containing treatments for osteopenia and osteoporosis embodying the principles of the disclosed subject matter.

FIG. 3 is a plan view of a bi-fold booklet packaging for dispensing gels, lozenges, and packets containing treatments for osteopenia and osteoporosis embodying the principles of the disclosed subject matter.

### DETAILED DESCRIPTION

### I. Introduction and Environment

As required, detailed aspects of the disclosed subject matter are disclosed herein; however, it is to be understood that the disclosed aspects are merely exemplary of the invention, which may be embodied in various forms. Therefore, specific structural and functional details disclosed herein are not to be interpreted as limiting, but merely as a basis for the claims and as a representative basis for teaching one skilled in the art how to variously employ the present invention in virtually any appropriately detailed structure.

### II. Embodiment or Aspect of the Treatment System

In accordance with the treatment system and method of the disclosed subject matter it has now been surprisingly found that preparations can be obtained consisting of administering a therapeutically effective amount of non-synthetic, bio-available hormones, vitamins, minerals, and agents to a subject at risk or suffering from osteopenia or osteoporosis for effective treatment thereof. Surprisingly the method improves the effectiveness and bioavability of preparations used in the treatment of osteopenia or osteoporosis by enhancing hormone, mineral, and agent absorption and action, and improving patient compliance.

A patient's physiology may dictate their response and effectiveness of any one treatment for osteopenia or osteoporosis. Therefore, the treating physician may need to use the following non-synthetic, bio-available hormones, vitamins, minerals, and agents alone or in combination to attain effective treatment of the condition. The hormones used for treatment include, but are not limited to thyroid hormones (e.g., thyroxine, or triiodothyronine), progesterone, calcitonin, estrogen (e.g., estradiol), dehydroepiandrosterone (DHEA), and natural testosterone. The vitamins used for treatment include vitamin K (e.g., menaquinone-4 (MK-4), or menaquinone-7 (MK-7)), vitamin D3 (e.g., cholecalciferol, or calcitrol), and vitamin B (e.g., thiamine (B1), riboflavin (B2), niacin (B3), pyridoxine (B6), folic acid (B9), or cobalamins (B12)). The minerals used for treatment include, but are not limited to iodine or iodide, zinc picolate, manganese, boron, strontium chloride, phosphorous, copper, calcium (e.g., calcium citrate, calcium gluconate, or calcium oxalate), magnesium (e.g., magnesium glycinate, or magnesium sulphate), and calcium hyroxyapatite. The agents used for treatment include, but are not limited to arginine, and wild yam extract as pre testosterone and progesterone agonists. The availability of the aforementioned hormones, vitamins, minerals, and agents may be by a prescription or non-prescription source.

The mode of administration the hormones, vitamins, minerals, and agents listed above can be customized for each patient based on patient preference and for achieving optimal bioavailability. The hormones, vitamins, minerals, and agents may be formulated in a variety of dosage forms, including, but not limited to liquids (e.g., oral solutions, nasal sprays, or suspensions), powders, articles for transdermal absorption (e.g., transdermal patch, impregnation matrices, gels, or creams), and ingested articles (e.g., capsules, lozenges, or tablets). The concentration of the hormones, vitamins, minerals and agents in their respective dosage forms will be determined by the physician as particular modes of administration are selected by the physician or patient. The hormone, vitamin, mineral, and agent preparations may include additional ingredients including, but not limited to pharmaceutically acceptable carriers, binders, coatings, and diluents as know to those skilled in the art of hormone, mineral, and agent delivery. The frequency in which the patient takes the hormones, vitamins, minerals, and agents may be determined by the physician.

The effective amount of hormone, vitamins, minerals and agents administered will depend upon the presence or absence of osteopenia or osteoporosis in the patient, the present severity of the aforementioned diseases, the desired therapeutically effective amount of active ingredient in the composition, and the particular dosage form desired.

Administration of thyroxine in accordance with the present invention will suitably comprise administration of dosages of from about 30 to about 60 micrograms (mcg). The formulation of thyroxine in the aforementioned dosages may be in the form of a powder, oral solution, nasal spray, patch, gel, cream, capsule, lozenge, or tablet.

Administration of triiodothyronine in accordance with the present invention will suitably comprise administration of dosages of from about 3.5 to about 7.5 mcg. The formulation of triiodothyronine in the aforementioned dosages may be in the form of a compounded powder, oral solution, nasal spray, patch, gel, cream, capsule, lozenge, or tablet.

Administration of progesterone in accordance with the present invention will suitably comprise administration of dosages of from about 25 to about 50 milligrams (mg). The formulation of progesterone in the aforementioned dosages may be in the form of a gel, cream, capsule, lozenge, or tablet.

Calcitonin is a 32-amino-acid polypeptide hormone that lowers plasma calcium and phosphate levels, and inhibits bone reabsorption (bone loss due to osteoclastic activity). Administration of calcitonin in accordance with the present invention will suitably comprise administration of dosages of about 250 mcg. The formulation of calcitonin in the aforementioned dosages may be in the form of a powder, oral solution, patch, gel, cream, capsule, lozenge, or tablet.

Estradiol is an estrogen used in the treatment of postmenopausal osteoporosis. Administration of estradiol in accordance with the present invention will suitably comprise administration of dosages of from about 0.25 to about 1.0 mg. The formulation of estradiol in the form of a powder, oral solution, or nasal spray will preferably comprise administration of a dosage of about 0.25 mg. The formulation of estradiol in the form of a patch, gel, cream, or lozenge will preferably comprise administration of a dosage of 0.25 mg, 0.375 mg, 0.5 mg, 0.75 mg, or 1.0 mg.

DHEA is an endogenous steroid that may be metabolized to produce testosterone, and estrogens. Administration of DHEA in accordance with the present invention will suitably comprise administration of dosages of about 5 mg. The formulation of DHEA in the aforementioned dosage may be in the form of a patch, gel, cream, capsule, lozenge, or sublingual tablet.

Natural testosterone aids in the retention of nitrogen, calcium, phosphorus, and potassium, all essential for maintenance of bone tissue in males. Administration of natural testosterone in accordance with the present invention will suitably comprise administration of dosages of from about 1 to about 5 mg. The formulation of natural testosterone in the aforementioned dosages may be in the form of a patch, gel, or cream.

Iodine and iodide are necessary for the synthesis of thyroid hormones (thyroxine and triiodothyronine) which regulates the metabolic rate in all cells. Administration of iodine and iodide in accordance with the present invention will suitably comprise administration of dosages of about 12.5 mg. The formulation of iodine or iodide in the aforementioned dosage may be in the form of a patch, gel, cream, capsule, lozenge, or tablet.

Zinc forms the essential part of many enzymes and plays an important role in protein synthesis and cell division. Administration of zinc picolate in accordance with the present invention will suitably comprise administration of dosages of about 12.5 mg. The formulation of zinc picolate in the aforementioned dosage may be in the form of a patch, gel, cream, capsule, lozenge, or tablet.

Manganese is necessary for the synthesis of mucopolysaccharides and activates a number of enzymes. Administration of manganese in accordance with the present invention will suitably comprise administration of dosages of about 1.0 mg. The formulation of manganese in the aforementioned dosage may be in the form of a patch, gel, cream, capsule, lozenge, or tablet.

Boron can aid in bone growth, improve bone-building osteoblasts, and boost bone density. Administration of boron in accordance with the present invention will suitably comprise administration of dosages of about 1.0 mg. The formulation of boron in the aforementioned dosage may be in the form of a patch, gel, cream, capsule, lozenge, or tablet.

Strontium chloride is a calcium analogue that aids in osteogenesis. Administration of strontium chloride in accordance with the present invention will suitably comprise administration of dosages of about 2.0 mg. The formulation of strontium chloride in the aforementioned dosage may be in the form of a patch, gel, cream, capsule, lozenge, or tablet.

Vitamin K increases bone mass, promotes osteotrophic processes, and slows osteoclastic process via calcium bonding. Administration of MK-4 or MK-7 in accordance with the present invention will suitably comprise administration of dosages of from about 10 to about 25 mg. The formulation of MK-4 or MK-7 in the aforementioned dosages may be in the form of a patch, gel, cream, capsule, lozenge, or tablet.

Phosphorous is a major component of the mineralization phase of bone. Administration of phosphorous in accordance with the present invention will suitably comprise administration of dosages of about 2.0 mg. The formulation of phosphorous in the aforementioned dosage may be in the form of a patch, gel, cream, capsule, lozenge, or tablet.

A deficiency in copper results in bone loss. Administration of copper in accordance with the present invention will suitably comprise administration of dosages of from about 2 to about 5 mg. The formulation of copper in the aforementioned dosages may be in the form of a patch, gel, cream, capsule, lozenge, or tablet.

Calcium combines with phosphorus to form calcium phosphate, a necessary composition of bone. Administration of sources of calcium comprising calcium citrate, calcium gluconate, or calcium oxalate in accordance with the present invention will suitably comprise administration of dosages of from about 500 to about 2,000 mg. Hydroxylapatite or hydroxyapatite is found in bone matrix and give bone rigidity. Administration of calcium hydroxyapatite, a source of calcium, in accordance with the present invention will suitably comprise administration of dosages of from about 1,000 to about 5,000 mg. The formulation of calcium citrate, calcium gluconate, calcium oxalate, or calcium hydroxyapatite in the aforementioned dosages may be in the form of a patch, gel, cream, capsule, lozenge, or tablet.

Magnesium and its salts are required for the activity of many enzymes. Administration of magnesium glycinate or magnesium sulphate in accordance with the present invention will suitably comprise administration of dosages of from about 500 to about 1,000 mg. The formulation of magnesium glycinate or magnesium sulphate in the aforementioned dosages may be in the form of a patch, gel, cream, capsule, lozenge, or tablet.

Vitamin D3 is used for the treatment of vitamin D deficiencies as a result of low intake or impaired absorption of the vitamin. Administration of cholecalciferol, or calcitrol in accordance with the present invention will suitably comprise administration of dosages of about 1,000 to about 50,000 international units (IU). The formulation of cholecalciferol, or calcitrol in the aforementioned dosages may be in the form of a patch, gel, cream, capsule, lozenge, or tablet.

Wild yam possesses dheydroepiandrosterone (DHEA) like properties and acts as a precursor to human sex hormones such as estrogen and progesterone. Administration of wild yam extract in accordance with the present invention will suitably comprise administration of dosages of about 25.0 mg. The formulation of wild yam extract in the aforementioned dosage may be in the form of a powder, oral solution, patch, gel, cream, capsule, lozenge, or tablet.

Arginine is an amino acid that can reduce the healing time of bone injuries. Administration of arginine in accordance with the present invention will suitably comprise administration of dosages of about 500.0 mg. The formulation of arginine in the aforementioned dosage may be in the form of a powder, oral solution, patch, gel, cream, capsule, lozenge, or tablet.

B vitamins are essential for building healthy bones. Administration of B1, B2, B3, B6, B9, or B12 in accordance with the present invention will suitably comprise administration of dosages of about 25 to about 100 mg. The formulation of B1, B2, B3, B6, B9, or B12 in the aforementioned dosages may be in the form of a powder, oral solution, patch, gel, cream, capsule, lozenge, or tablet.

For the purposes of liquid administration (e.g., oral solutions, nasal sprays, or suspensions), the preparations of the present invention will preferably be put in a container provided with a means of enabling application of the preparation to the target site. Suitable applicators are known in the art and include those adapted for administration of liquid compositions in their respective forms. For the purpose of powders and articles for transdermal absorption (e.g., transdermal patch, impregnation matrices, gels, or creams), the preparations of the present invention will preferably utilize a means of enabling application and adsorption of the preparation to the target site. Suitable vehicles include the aforementioned patches, impregnation matrices, gels, or creams, and other vehicles known in the art. For the purpose of ingested articles (e.g., capsules, lozenges, or tablets), the preparations will preferably be put in a container enabling proper dispensing of the preparation.

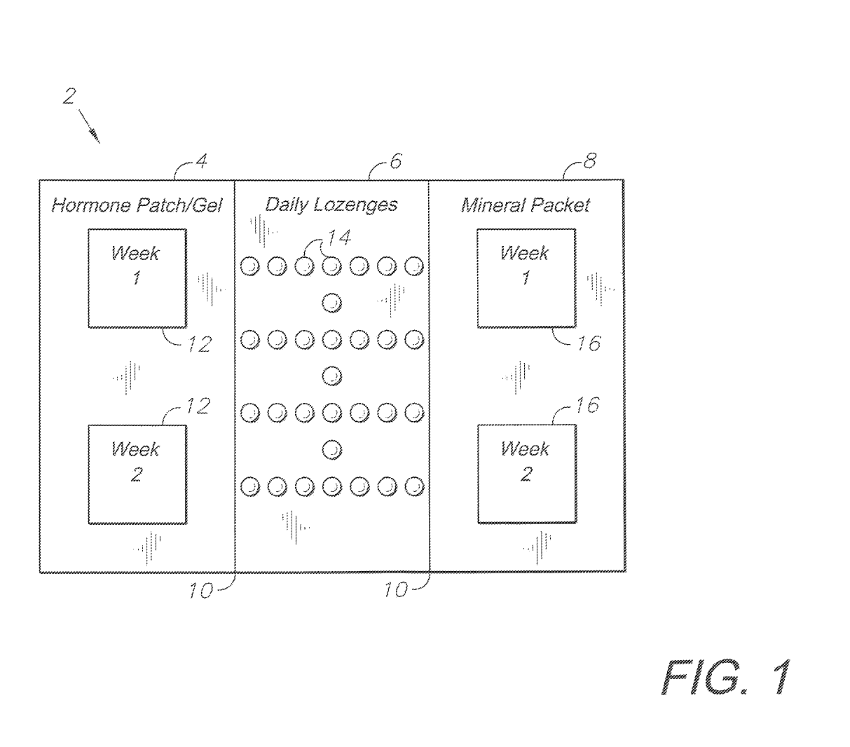

Referring to FIG. 1, the reference numeral 2 generally designates a type of packaging or container for dispensing various formulations of the hormones, vitamins, minerals, and agents disclosed in this application. A tri-fold packaging 2, manufactured from paper or plastic, is shown with a first panel 4, second panel 6, and third panel 8. The first panel 4 dispenses plastic or foil packets 12 containing, but not limited to oral solutions, patches, gels, or creams. A second panel 6 has a plurality of foil-backed blister pockets 6 for dispensing ingested articles including, but not limited to capsules, lozenges, or tablets. A third panel 8 dispenses plastic or foil packets 16 containing capsules, lozenges, or tablets. The frequency in which the patient takes the packets, ingested articles, and foil packets may be determined by a physician. The tri-fold packaging 2 contains seams 10 for folding the packaging 2 so it can be stored in the pocket of clothing or in a bag.

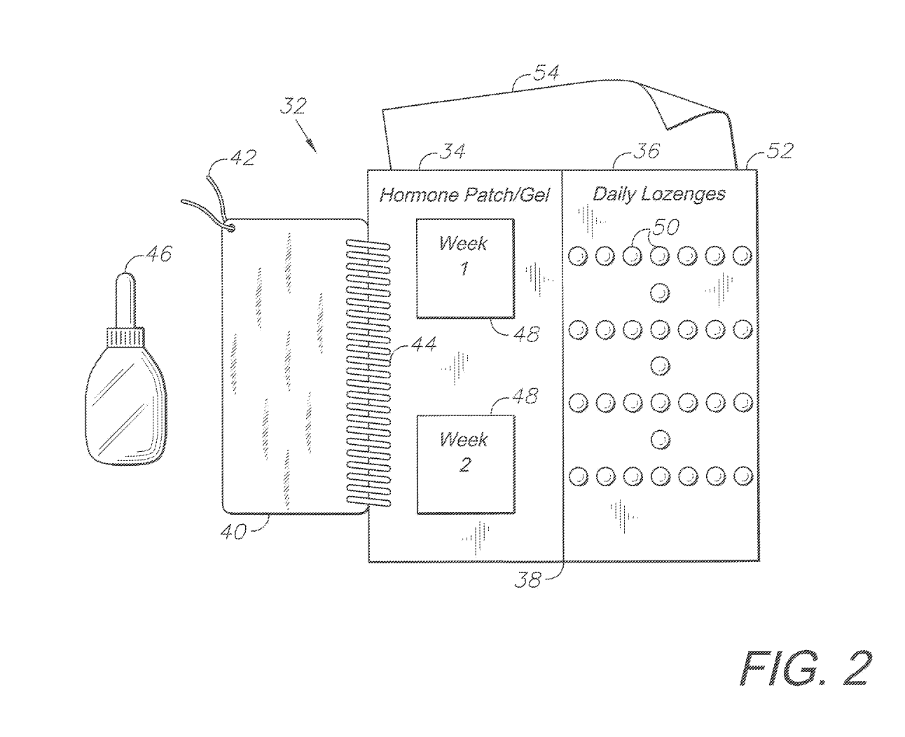

Referring to FIG. 2, the reference numeral 32 generally designates an alternative type of packaging for dispensing various formulations of the hormones, vitamins, minerals, and agents disclosed in this application. A bi-fold packaging 32 has a first panel 34 and second panel 36 and an attached pouch 40. The first panel 34 dispenses plastic or foil packets 48 containing, but not limited to oral solutions, patches, gels, or creams. Alternatively, the first panel 34 can dispense plastic or foil packets containing capsules lozenges, or tablets. A second panel 36 has a plurality of foil-backed blister pockets 50 for dispensing ingested articles including, but not limited to capsules, lozenges, or tablets. The pouch 40 is attached to one of the panels 34, 36 by an attachment, such as a plastic loop attachment 44. The pouch 40 securely holds a liquid container 46 such as, but not limited to a nasal spray bottle for holding nasal spray solutions, or dropper bottle for holding oral solutions. A drawstring 42 encircles the top of the pouch 40 for securing the liquid container 46 therein. A pocket 52 running the length of the first and second panels 34, 36 contains printed instructions for patient on use of the preparations contained within the tri-fold packaging 32. The frequency in which the patient takes the packets, ingested articles, and the liquids in the liquid container 46 may be determined by a physician. The bi-fold packaging 32 contains a seam 38 for folding the packaging 32 so it can be stored in the pocket of clothing or in a bag.

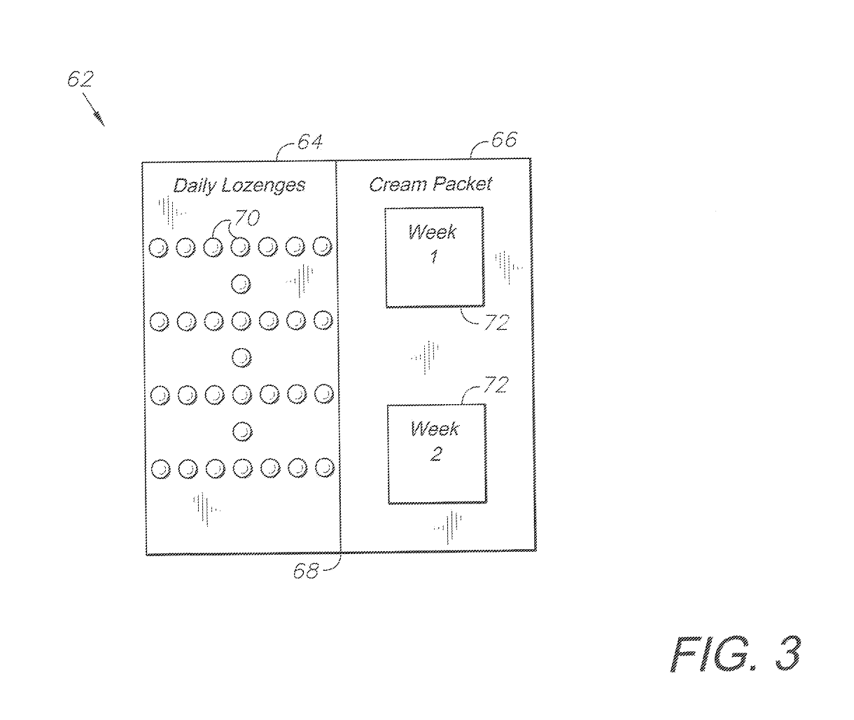

Referring to FIG. 3, the reference numeral 62 generally designates an alternative type of packaging for dispensing various formulations of the hormones, vitamins, minerals, and agents disclosed in this application. The bi-fold packaging 62 has a first panel 64 and a second panel 66 that may be folded along a seam 68. The first panel has a plurality of foil-backed blister pockets 70 for dispensing ingested articles including, but not limited to capsules, lozenges, or tablets. The second panel 66 dispenses plastic or foil packets 72 containing a cream comprising vitamins and minerals for topical application. The frequency in which the patient takes the ingested articles and packets may be determined by a physician.

### II. Embodiment or Aspect of the Treatment Method

A method embodying the present invention comprises diagnosis of a patient presenting signs of, or suffering from, osteopenia or osteoporosis, and evaluation of a treatment plan for treating a patient using the system described above. Upon diagnosis of the presence and severity of osteopenia or osteoporosis by a physician or healthcare professional, a treatment plan is selected by the physician and patient. Utilizing the hormones, vitamins, minerals, and agents described above, the physician selects the appropriate dosage, formulation, and frequency of administration. During routine evaluations of the patient, the physician can adjust the individual hormones, vitamins, minerals, and agents used to treat the patient, and the dosage, formulation, and frequency of the components to maximize therapeutic effect, bioavailability, and patient comfort and compliance with their treatment of osteopenia or osteoporosis.

It will be appreciated that the components of the treatment method can be used for various other applications. Moreover, the packaging 2, 32 can be fabricated in various sizes and from a wide range of suitable materials, using various manufacturing and fabrication techniques.

It is to be understood that while certain aspects of the disclosed subject matter have been shown and described, the disclosed subject matter is not limited thereto and encompasses various other embodiments and aspects.

Having thus described the invention, what is claimed as new and desired to be secured by Letters Patent is:

1. A system for treating a patient suffering from osteopenia and/or osteoporosis, wherein a physician has established a treatment plan for the patient, the system comprising:
a plurality of hormone applications comporting with the treatment plan, said hormone application being arranged by dosage;
a plurality of mineral applications comporting with the treatment plan, said mineral applications being arranged by dosage;
a plurality of vitamin applications comporting with the treatment plan, said vitamin applications being arranged by dosage;
a storage unit adapted for packaging the said of hormone applications, said mineral applications, and said vitamin applications; and
said storage unit being adapted for dispensing said hormone applications, said mineral applications, and said vitamin applications.

2. The system of claim 1, further comprising;
said hormone applications comprised of non-synthetic, bio-available hormones;
said mineral applications comprised of non-synthetic, bio-available minerals; and
said vitamin applications comprised of non-synthetic, bio-available vitamins.

3. A method for treating a patient suffering from osteopenia and/or osteoporosis comprising the steps:
diagnosing the patient;
establishing a treatment plan for said patient, wherein said treatment plan includes the administration of hormone applications, mineral applications, and vitamin applications;
providing a plural a plurality of hormone applications comporting with the treatment plan, said hormone application being arranged by dosage;
providing a plurality of mineral applications comporting with the treatment plan, said mineral applications being arranged by dosage;
providing a plurality of vitamin applications comporting with the treatment plan, said vitamin applications being arranged by dosage;
providing a storage unit adapted for packaging the said of hormone applications, said mineral applications, and said vitamin applications;
storing said the said of hormone applications, said mineral applications, and said vitamin applications within said storage unit; and
dispensing said hormone applications, said mineral applications, and said vitamin applications from said storage unit.
